(19) United States
(12) Patent Application Publication
(10) Pub. No.: US 2014/0194699 A1
(43) Pub. Date: Jul. 10, 2014

(54) SINGLE PORT SURGICAL ROBOT AND CONTROL METHOD THEREOF

(71) Applicant: SAMSUNG ELECTRONICS CO., LTD., Suwon-si (KR)

(72) Inventors: Se-Gon ROH, Suwon-si (KR); Young-Do Kwon, Yongin-si (KR); Youn-Baek Lee, Suwon-si (KR); Yong-Jae Kim, Seoul (KR); Jeong-Hun Kim, Hwaseong-si (KR); Kyung-Shik Roh, Seongnam-si (KR); Kyung-won Moon, Yongin-si (KR); Tae-Jun Sang, Suwon-si (KR); Jong-Won Lee, Euiwang-si (KR); Byung-June Choi, Kunpo-si (KR); Tae-Sin Ha, Seongnam-si (KR)

(73) Assignee: SAMSUNG ELECTRONICS CO., LTD., Suwon-si (KR)

(21) Appl. No.: 13/935,841

(22) Filed: Jul. 5, 2013

(30) Foreign Application Priority Data
Jan. 8, 2013 (KR) 10-2013-0002277

(51) Int. Cl. A61B 19/00; A61B 17/34

(52) U.S. Cl. CPC A61B 19/2203; A61B 19/5202; A61B 19/5212; A61B 17/34; A61B 19/5225
USPC 600/249

(57) ABSTRACT

A single port surgical robot capable of setting the location of a remote center motion (RCM) point, and a control method thereof includes: a first link connected to a body by a first joint in a direction perpendicular to the body, the first link having a linear structure; a second link connected to the upper end of the first link by a second joint, the second link having a curved structure; a third link connected to the upper end of the second link by a third joint, the third link having a cylindrical structure; a plurality of light-emitting units arranged on the lower end of the third link along the circumference of the third link, and configured to emit light toward a remote center motion (RCM) point; and a controller configured to adjust the location of the RCM point.

### CROSS-REFERENCE TO RELATED APPLICATIONS

This application claims the benefit of Korean Patent Application No. 10-2013-0002277, filed on Jan. 8, 2013 in the Korean Intellectual Property Office, the disclosure of which is incorporated herein by reference.

### BACKGROUND

1. Field

One or more embodiments relate to a single port surgical robot and a control method thereof, and more particularly, to a single port surgical robot capable of setting the location of a remote center motion (RCM) point, and a control method thereof.

2. Description of the Related Art

Minimal invasive surgery is the collective term for surgery capable of reducing the size of an affected area. Unlike a laparotomy that is performed after making a large operation incision in a part (for example, the abdomen) of a human body, the minimal invasive surgery is surgery of making one or more small slits (or at least one invasive hole) having a size of approximately 0.5 cm to 1.5 cm in the abdomen, inserting a video camera and surgical instruments in the abdominal cavity through the slits, and then performing operation while viewing video.

The minimal invasive surgery has potential benefits of less pain after surgery, faster recovery of bowel movements, faster ingestion of foods, shorter hospital stays, faster recovery to normal conditions, and good cosmetic results due to a small slit, compared to the laparotomy. Because of the benefits, the minimal invasive surgery has been used, for example, in cholecystectomy, prostate cancer, hernia repair, etc. and is extending its use to more various fields.

A surgical robot that is currently used in minimal invasive surgery includes a master console and a slave robot. The master console generates control signals according to a physician's manipulations, and transfers the control signals to the slave robot. The slave robot receives the control signals from the master console, and applies manipulations required for surgery to a patient according to the control signals.

The slave robot includes at least one robot arm, and robotic surgical instruments are mounted on the upper end of each robot arm. The robotic surgical instruments are inserted into the patient's body through an incision point. The robot arm is located outside the incision point, and functions to fix the positions and orientations of the robotic surgical instruments while surgery is being performed.

The robotic surgical instruments can be controlled such that they operate only within a conical workspace whose vertex is a virtual central point set to a predetermined location. The virtual central point is called a “remote center motion (RCM) point.” If the RCM point is exactly positioned at the patient's incision point, the robotic surgical instruments will move only within a conical workplace in the patient's body even when there is movement of the robot arm located outside the incision point. Accordingly, it is possible to prevent the patient's incision point from being damaged due to the robot arm's movement.

### SUMMARY

Therefore, it is an aspect of one or more embodiments to provide a single port surgical robot capable of setting the location of a remote center motion (RCM) point, and a control method thereof.

Additional aspects and/or advantages of one or more embodiments will be set forth in part in the description which follows and, in part, will be apparent from the description, or may be learned by practice of one or more embodiments of disclosure. One or more embodiments are inclusive of such additional aspects.

In accordance with one or more embodiments, a single port surgical robot may include: a first link connected to a body by a first joint in a direction perpendicular to the body, the first link having a linear structure; a second link connected to the upper end of the first link by a second joint, the second link having a curved structure; a third link connected to the upper end of the second link by a third joint, the third link having a cylindrical structure; a plurality of light-emitting units arranged on the lower end of the third link along the circumference of the third link, and configured to emit light toward a remote center motion (RCM) point; and a controller configured to adjust the location of the RCM point.

In accordance with one or more embodiments, a control method of a single port surgical robot may include: operating a plurality of light-emitting units arranged along the circumference of a cylindrical link, and configured to emit light toward a remote center motion (RCM) point; and adjusting the location of the RCM point.

Since a RCM point is indicated using a plurality of light-emitting units, a user can recognize the location of the RCM point.

Also, since a user can recognize the location of a RCM point, it is possible to set the location of the RCM point, and possibly improve accuracy of the set location of the RCM point.

### BRIEF DESCRIPTION OF THE DRAWINGS

These and/or other aspects of the invention will become apparent and more readily appreciated from the following description of embodiments, taken in conjunction with the accompanying drawings of which:

FIG. 1 is a perspective view showing the appearance of a master console of a single port surgical robot according to one or more embodiments;

FIG. 2 is a perspective view showing the appearance of a slave robot of a single port surgical robot according to one or more embodiments;

FIG. 3 is a perspective view showing the internal configuration of a slave robot according to one or more embodiments, such as the slave robot shown in FIG. 2;

FIG. 4 shows a structure with three light-emitting units of emitting different colors of light according to one or more embodiments;

FIG. 5 shows a structure with two light-emitting units of emitting different colors of light according to one or more embodiments;

FIG. 6 shows a remote center motion (RCM) point configured with three light-emitting units respectively having different shapes of pointers according to one or more embodiments;

FIG. 7 shows a RCM point configured with two light-emitting units respectively having different shapes of pointers according to one or more embodiments;

FIGS. 8A through 8F are views for explaining a method of setting the location of a RCM point in a slave robot according to one or more embodiments, such as the slave robot shown in FIG. 2;

FIG. 9 is a block diagram showing the control configuration of a slave robot according to one or more embodiments;

FIG. 10 is a block diagram showing the control configuration of a slave robot according to one or more embodiments;

FIG. 11 is a flowchart showing a control method of a slave robot according to one or more embodiments, such as the slave robot shown in FIG. 9 or 10;

FIG. 12 is a block diagram showing the control configuration of a slave robot according to one or more embodiments;

FIG. 13 is a view for explaining a process of setting the location of a RCM point in a slave robot according to one or more embodiments, such as the slave robot shown in FIG. 12, wherein images photographed by a photographing unit are arranged in the order of time; and

FIG. 14 is a flowchart showing a control method of a slave robot according to one or more embodiments, such as the slave robot shown in FIG. 12.

### DETAILED DESCRIPTION

Reference will now be made in detail to one or more embodiments, illustrated in the accompanying drawings, wherein like reference numerals refer to like elements throughout. In this regard, embodiments of the present invention may be embodied in many different forms and should not be construed as being limited to embodiments set forth herein, as various changes, modifications, and equivalents of the systems, apparatuses and/or methods described herein will be understood to be included in the invention by those of ordinary skill in the art after embodiments discussed herein are understood. Accordingly, embodiments are merely described below, by referring to the figures, to explain aspects of the present invention.

The present invention disclosed herein may be applied to a single port surgical robot. The single port surgical robot is a surgical robot which performs surgery after inserting a plurality of robotic surgical instruments in a patient's abdominal cavity through an incision point. The single port surgical robot can be considered as a concept distinct from a multi-port surgical robot which performs surgery after inserting a plurality of robotic surgical instruments in a patient's abdominal cavity through a plurality of incision points.

The single port surgical robot includes a master console and a slave robot. The master console generates control signals according to an operator's manipulations, and transfers the control signals to the slave robot. The slave robot receives the control signals from the master console, and moves according to the control signals to apply manipulations required for surgery to a patient. Here, the operator is a medical professional such as a medical specialist or a doctor. Or the operator may be a person qualified or authorized as a medical professional. In a broad sense, the operator may include a user who controls the operation of a single port surgical robot.

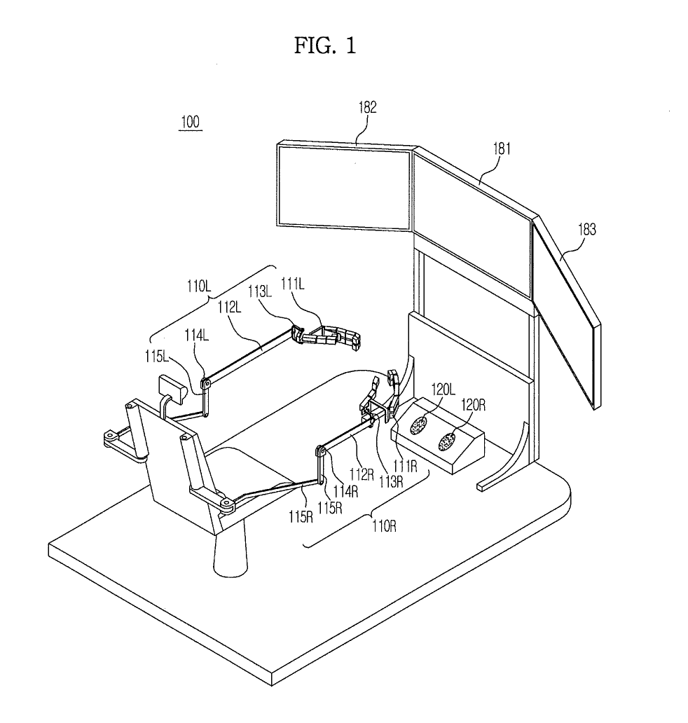

Hereinafter, the master console will be described in more detail with reference to FIG. 1.

FIG. 1 is a perspective view showing the appearance of a master console 100 of a single port surgical robot. As shown in FIG. 1, the master console 100 may include input units 110R, 110L, 120R, and 120L and display units 181, 182, and 183.

The input units 110R, 110L, 120R, and 120L may receive commands for remotely manipulating the operation of a slave robot, from an operator. In order to receive commands from the operator, the input units 110R, 110L, 120R, and 120L may include, for example, at least one among haptic devices, clutch pedals, and buttons. FIG. 1 shows the case in which the input units 110R, 110L, 120R, and 120L include two haptic devices (110R and 110L) and two clutch pedals (120R and 120L).

The two haptic devices 110R and 110L may be disposed in the left and right sides of a chair on which the operator sits. The haptic devices 110R and 110L may include end-effectors 111R and 111L, support links 112R and 112L, and one or more connection links 115R and 115L disposed between the support links 112R and 112L and the chair.

The end-effectors 111R and 111L are parts that the operator's hands contact. For example, at least one among the palms, backs, and fingers of the operator's hands may contact the end-effectors 111R and 111L. For this, the end-effectors 111R and 111L may include one or more multi-joint robot fingers. The multi-joint robot fingers may be in an arrangement similar to a human's fingers. FIG. 1 shows the case in which three multi-joint robot fingers are respectively arranged at positions corresponding to the thumb, index finger, and middle finger of a human's hand.

FIG. 1 shows an example in which three multi-joint robot fingers are provided in each of the end-effectors 111R and 111L, however, the number or positions of the multi-joint robot fingers are not limited. For example, each of the end-effectors 111R and 111L may include multi-joint robot fingers more or less than three, and the multi-joint robot fingers may be arranged at positions corresponding to at least one among the thumb, index finger, middle finger, ring finger, and little finger of a human's hand.

FIG. 1 shows the case in which a plurality of multi-joint robot fingers are provided in each of the end-effectors 111R and 111L, however, the shapes of the end-effectors 111R and 111L are not limited to multi-joint robot fingers. For example, the end-effectors 111R and 111L may be in the shape of a pencil or stick which an operator can hold with his or her hand. As another example, the end-effectors 111R and 111L may be in the shape of scissors which an operator can use with his or her at least two fingers.

FIG. 1 shows the case in which the left end-effector 111L and the right end-effector 111R have the same shape, however, the left and right end-effectors 111L and 111R may have different shapes. For example, the left end-effector 111L may have a scissors shape, and the right end-effector 111R may include at least one articulated robot finger.

The multi-joint robot finger may include a plurality of links and a plurality of joints. The joints are connection parts between links, and may have at least 1 degree of freedom (DOF). Here, the DOF means a degree of freedom in Kinematics or Inverse Kinematics. The DOF of an instrument is the number of independent motions of the instrument, or the number of variables that decide independent motions with respect to relative positions between links. For example, an object which is placed in a 3-dimensional space consisting of x-axis, y-axis, and z-axis has at least one DOF of 3 DOFs (locations on the individual axes) for deciding the spatial location of the object, and 3 DOFs (rotation angles with respect to the individual axes) for deciding the spatial orientation of the object. If the object is movable along the individual axes and rotatable with respect to the individual axes, the object can be understood to have 6 DOFs.

In each joint of each multi-joint robot finger, a detector for detecting information associated with the state of the joint may be provided. The detector may include at least one of a force/torque detector for detecting a force/torque applied to the corresponding joint, a location detector for detecting the location of the corresponding joint, and a velocity detector for detecting the velocity of the corresponding joint.

On the end of the multi-joint robot finger, for example, a ring-shaped loop may be provided. The operator may insert his or her fingertip into the ring-shaped loop. If the operator moves his or her finger after inserting the finger into the ring-shaped loop, the corresponding multi-joint robot finger moves in correspondence to the movement of the operator's finger, and a detector installed at each joint of the multi-joint robot finger detects information associated with the state of the corresponding joint.

The detected state information is transferred to a controller (not shown) of the master console 100. The controller of the master console 100 generates control signals for driving the slave robot based on the state information received from the detector. The generated control signals are transferred to the slave robot through a network. At this time, the network may be a wired network, a wireless network, or a wired-wireless combined network.

The support links 112R and 112L are mechanically connected to the end-effectors 111R and 111L. The support links 112R and 112L function to support the operator's body parts from the wrists to the elbows. The support links 112R and 112L may include wrist support units 113R and 113L and elbow support units 114R and 114L.

The wrist support units 113R and 113L are disposed at locations corresponding to the operator's wrists. The wrist support units 113R and 113L may have various shapes. For example, the wrist support units 113R and 113L may be in the shape of a ring. In this case, the operator inserts his or her at least one fingertip into the ring-shaped loops of the multi-joint robot fingers after passing his or her hands through the ring-shaped wrist support units 113R and 113L. As another example, the wrist support units 113R and 113L may have a semicircular structure. In this case, the opened parts of the semicircular structures may be positioned toward the inside, that is, toward the operator, and the curved parts of the semicircular structures may be positioned toward the outside. However, it is also possible that the curved parts of the semicircular structures are positioned toward the inside, and the opened parts of the semicircular structures are positioned toward the outside. Or, it is also possible that the curved parts of the semicircular structures face the ground, and the opened parts of the semicircular structures face in a direction perpendicular to the ground.

The elbow support units 114R and 114L are disposed at locations corresponding to the operator's elbows. The elbow support units 114R and 114L may have U-shaped structures. For example, the opened parts of the elbow support units 114R and 114L may face in a direction perpendicular to the ground, and the curved parts of the elbow support units 114R and 114L may face the ground. As another example, the opened parts of the elbow support units 114R and 114L may be positioned toward the inside or the outside. In this case, the elbow support units 114R and 114L may further include securing elements (not shown) for wrapping the operator's elbows and securing them to the elbow support units 114R and 114L.

The connection links 115R and 115L are disposed between the support links 112R and 112L and the chair, and function to mechanically connect the support links 112R and 112L to the chair. There are one or more connection links 115R and 115L. Each of joints is provided in connection part of the connection link 115R and 115R may have at least 1 DOF.

FIG. 1 shows a structure in which the support links 112R and 112L and the chair are connected by one or more connection links 115R and 115L, however, the structure of the master console 100 is not limited to the structure shown in FIG. 1. For example, the support links 112R and 112L and the one or more connection links 115R and 115L may be omitted although the end-effectors 111R and 111L are configured to include a plurality of multi-joint robot fingers as shown in FIG. 1. In this case, the end-effectors 111R and 111L may further include communication units (not shown) for transmitting/receiving data to/from the controller of the master console 100 through wired/wireless communication.

The display units 181, 182, and 183 display at least one piece of image data and surgery information. The image data that is displayed on the display units 181, 182, and 183 may be images photographed by an endoscope of the slave robot, or images obtained by image-processing the photographed images. The image processing may be at least one among image magnification, image reduction, image movement, image rotation, combine with other images, and filtering. The image processing may be performed by at least one of the slave robot and the master console 100.

There may be one or more display units 181, 182, and 183. FIG. 1 shows the case in which three display units 181, 182, and 183 are arranged in a line in the master console 100. For example, the display units 181, 182, and 183 may display different images. In detail, images photographed by an endoscope may be displayed on the main display 101 located in front of the operator. Also, information related to the operating state of the slave robot and information about the patient may be respectively displayed on the sub display units 182 and 183 located in the left and right side of the main display unit 181. As another example, the display units 181, 182, and 183 may display the same image. In this case, each of the display units 181, 182, and 183 may display the same image, or the display units 181, 182, and 183 may together display the same image.

The display units 181, 182, and 183 may be implemented as liquid crystal displays (LCDs) or light emitting displays (LEDs).

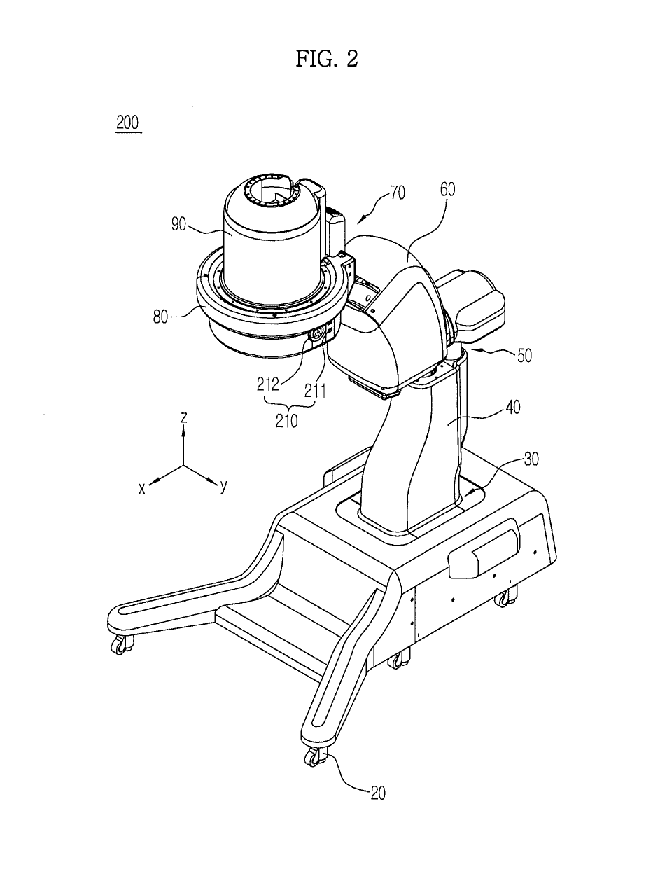

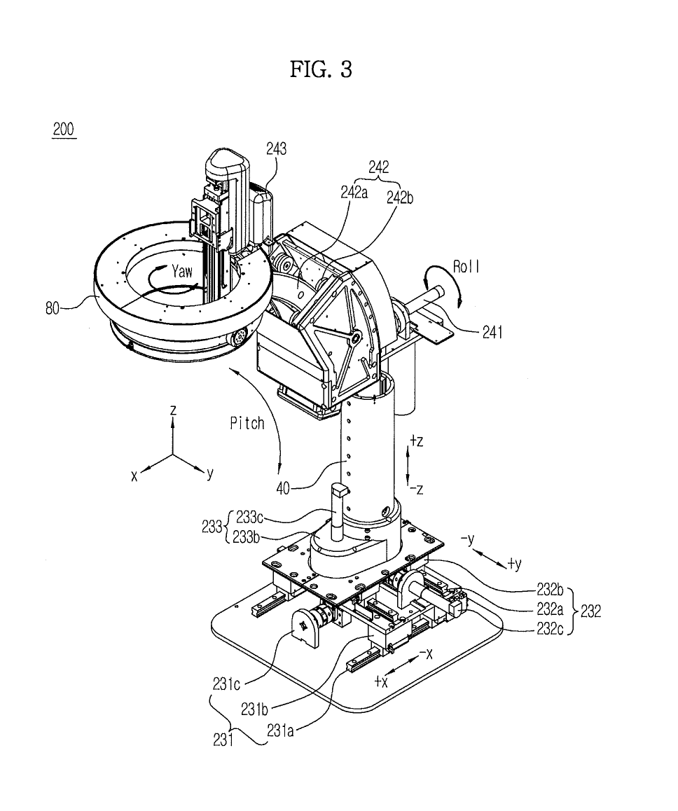

Now, the slave robot 200 will be described with reference to FIGS. 2 and 3. FIG. 2 is a perspective view showing the appearance of a slave robot 200 of a single port surgical robot, and FIG. 3 is a perspective view showing the internal configuration of the slave robot shown in FIG. 2.

As shown in FIGS. 2 and 3, the slave robot 200 may include a caster unit 20, a first link 40, a second link 60, a third link 80, a first joint 30, a second joint 50, a third joint 70, a surgical instrument assembly 90, and a manipulation unit 210.

The caster unit 20 is used to move the slave robot 200, and located on the bottom of the slave robot body. The caster unit 20 may include a plurality of casters. Each caster may include a lever (not shown) for changing the operating state of the caster. An operator adjusts the location of the lever to change the operating state of the caster. The operating state of the caster may include brake, free swivel, and directional lock (swivel lock).

The first link 40 is in the shape of a linear pillar, and positioned in a direction perpendicular to the body.

The first joint 30 is provided in the connection part of the body and the first link 40. The first joint 30 may be implemented as a prismatic joint movable along a designated axis of x-, y-, and z-axes. Since the first joint 30 is aimed at adjusting x, y, and z coordinates of a RCM point, the first joint 30 may have 3 DOFs. In detail, the first joint 30 has 3 DOFs including x-axis translation motion, y-axis translation motion, and z-axis translation motion. For this, the first joint 30 may include an x-axis driver 231, a y-axis driver 232, and a z-axis driver 233.

The x-axis driver 231 may include linear motion (LM) guides 231a and 231b, and a motor 231c providing driving force to the LM guides 231a and 231b. The LM guides 231a and 231b may be at least one LM rail 231a and at least one LM block 231b. FIG. 3 shows the case in which two LM rails 231a are arranged in parallel in the x-axis direction on a plate, and two LM blocks 231b are provided on each LM rail 231a. LM block 231b is moved along the LM rail 231a by a driving force provided from the motor 231c.

The y-axis driver 232 may include LM guides 232a and 232b, and a motor 232c providing driving force to the LM guides 232a and 232b. The LM guides 232a and 232b may be at least one LM rail 232a and at least one LM block 232b. FIG. 3 shows the case in which two LM rails 232a are arranged in parallel in the y-axis direction on a plate, and two LM blocks 232b are provided on each LM rail 232a. LM rails 232a of the y-axis driver 232 are located perpendicular to the LM rails 231a of the x-axis driver 231. A plate is disposed on the LM blocks 232b of the y-axis driver 232. The z-axis driver 233 is disposed on the plate.

The z-axis driver 233 may include a motor 233c for providing driving force, and at least one gear 233b connected to the first link 40 to transfer the driving force from the motor 233c to the first link 40.

The second link 60 may be mechanically connected to the upper end of the first link 40. The second link 60 has a curved shape as shown in FIG. 3. In detail, the second link 60 is in the shape of a part of a circular arc.

The second joint 50 is provided in the connection part of the first link 40 and the second link 60. The second joint 50 may be implemented as a revolute joint rotatable with respect to a designated axis among x-, y-, and z-axes. Since the second joint 50 is aimed at rotating the surgical instrument assembly 90, the second joint 50 may have 2 DOFs. In detail, the second joint 50 has 2 DOFs including roll-direction rotations and pitch-direction rotations of the surgical instrument assembly 90. For this, the second joint 50 may include a roll driver 241 and a pitch driver 242.

The roll driver 241 provides driving force to the second joint 50 according to an input signal received from the second manipulation unit 212 or a control signal received from the master console (see FIG. 1, 100) so that the surgical instrument assembly 90 can rotate in the roll direction. The roll driver 241 may be implemented as one of a motor, a vacuum pump, and a hydraulic pump.

The pitch driver 242 provides driving force to the second joint 50 according to the input signal received from the second manipulation unit 212 or the control signal received from the master console 100 so that the surgical instrument assembly 90 can rotate in the pitch direction. The pitch driver 242 may include R guides 242a and 242b for guiding circular motion of the second link 60, and a motor (not shown) for providing driving force to the R guides 242a and 242b.

The third link 80 is mechanically connected to the upper end of the second link 60. The third link 80 is in the shape of a cylinder as shown in FIG. 3. The surgical instrument assembly 90 is provided on the third link 80.

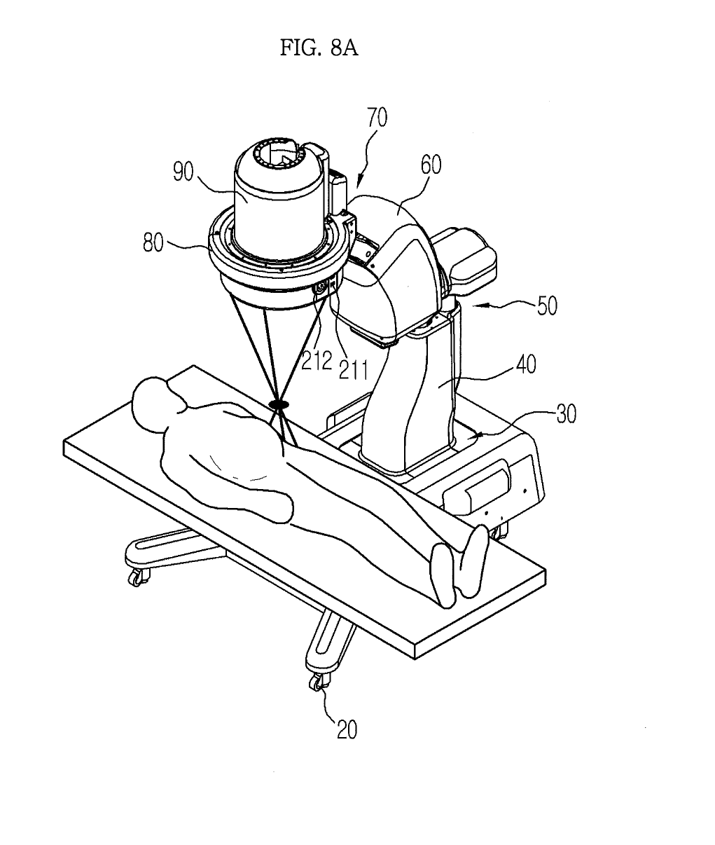

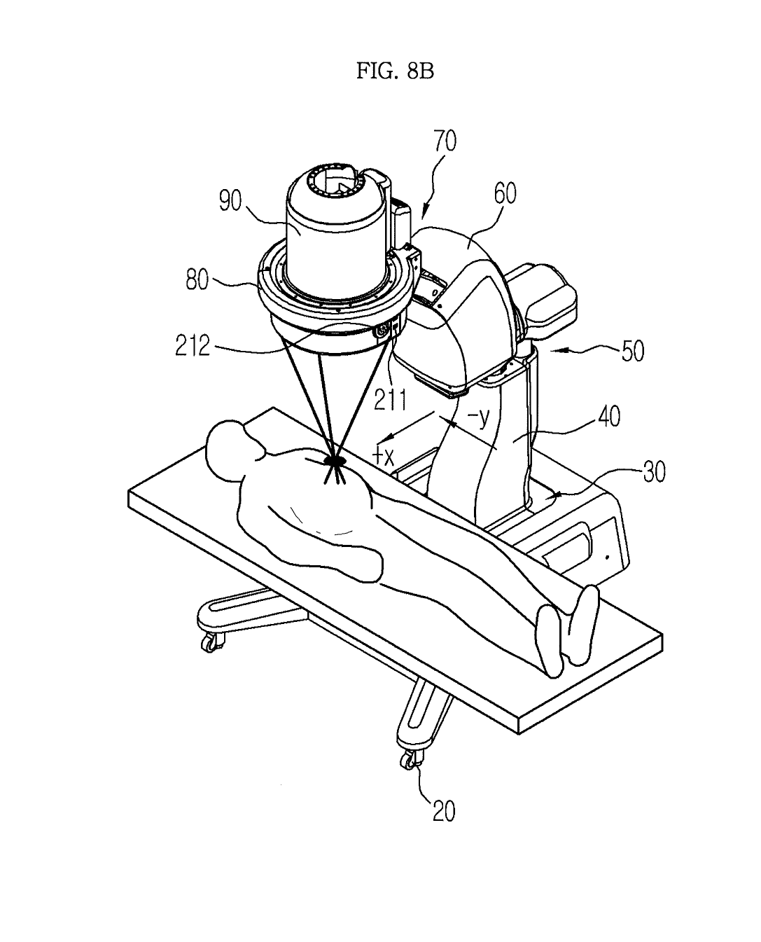

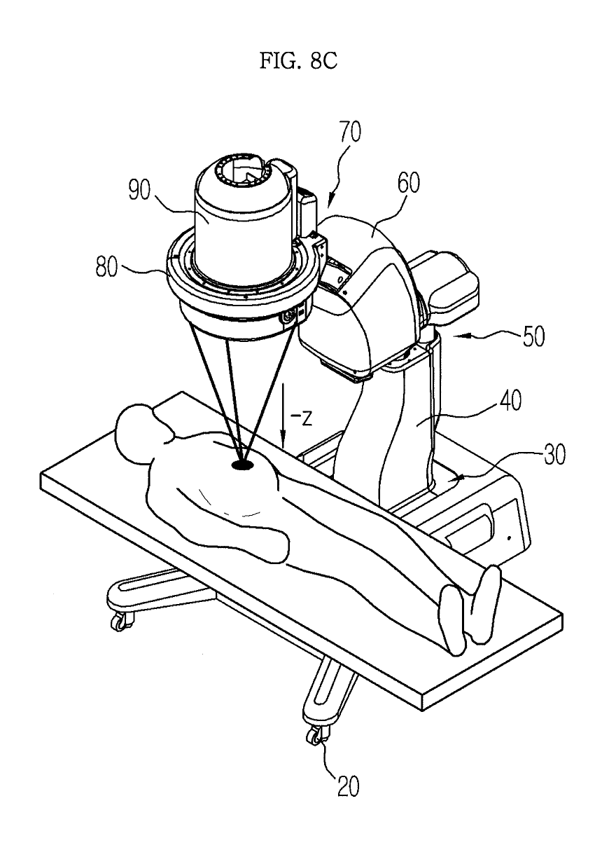

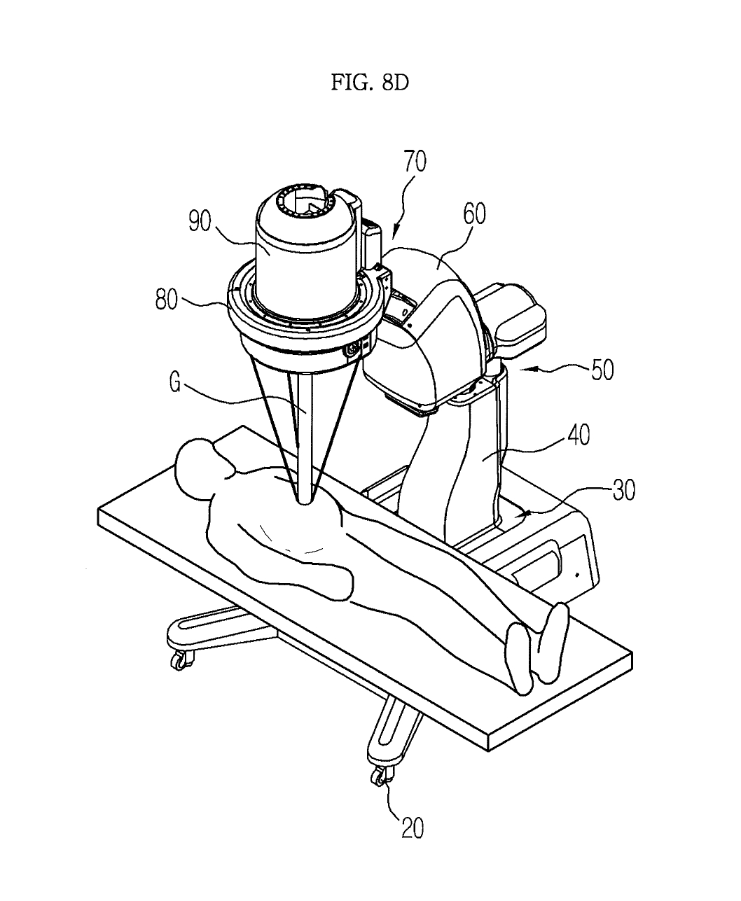

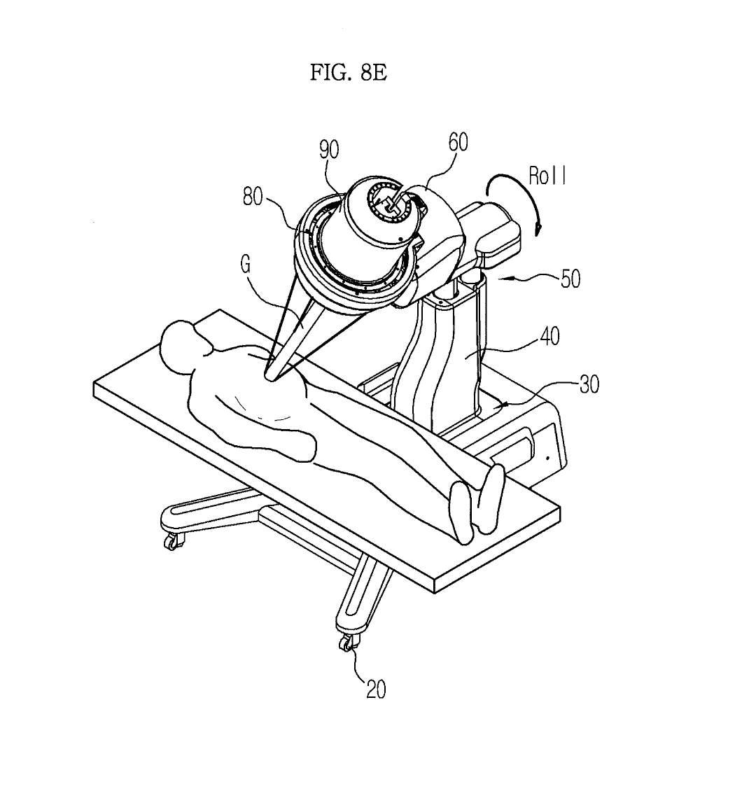

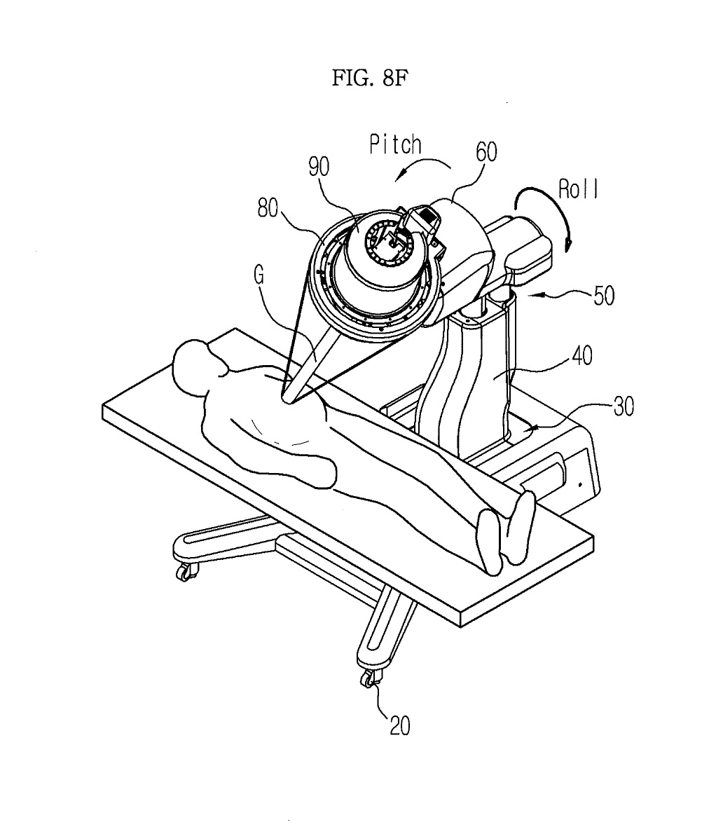

The surgical instrument assembly 90 includes a cylindrical casing, and a plurality of robotic surgical instruments arranged along the inner circumference of the casing. The robotic surgical instruments may include instruments for resecting or coagulating body tissues, and an endoscope for photographing the abdominal cavity. At least one robotic surgical instrument selected by an operator among the plurality of robotic surgical instruments arranged along the inner circumference of the casing may be inserted into a patient's abdominal cavity through a guide tube G (See FIG. 8D). The surgical instrument assembly 90 may be implemented to be mechanically separated from the third link 80. As such, if the surgical instrument assembly 90 is separated from the third link 80, it is easy to replace surgical instruments used in surgery with new ones or to disinfect the used surgical instruments.

The third joint 70 is provided in the connection part of the third link 80 and the second link 60. The third joint 70 may be implemented as a revolute joint rotatable with respect to a designated axis among x-, y-, and z-axes. Since the third joint 70 is aimed at rotating the surgical instrument assembly 90, the third joint 70 may have 1 DOF. In detail, the third joint 70 has 1 DOF including yaw-direction rotations of the surgical instrument assembly 90. For this, the second joint 50 may include a yaw driver 243.

The yaw driver 243 provides driving force to the third joint 70 according to the input signal received from the second manipulation unit 212 or the control signal received from the master console 100 so that the surgical instrument assembly 90 can rotate in the yaw direction. The yaw driver 243 may be implemented as one of a motor, a vacuum pump, and a hydraulic pump.

The manipulation unit 210 may allow the operator to input a command for setting the location of a RCM point of the slave robot 200. The manipulation unit 210 may include a first manipulation unit 211 and a second manipulation unit 212.

The first manipulation unit 211 may receive a command for switching the motion mode of the slave robot from the operator.

For example, the first manipulation unit 211 may be implemented as a two-phase switch. The two-phase switch may be set to one of first and second phases. If the two-phase switch is set to the first phase, the slave robot 200 may be set to a translation mode, and if the two-phase switch is set to the second phase, the slave robot 200 may be set to a rotation mode.

As another example, the first manipulation unit 211 may be implemented as a three-phase switch. The three-phase switch may be set to one of first, second, and third phases. If the three-phase switch is set to the first phase, the slave robot 200 may be set to a translation mode, and if the three-phase switch is set to the second phase, the slave robot 200 may be set to a rotation mode. Also, if the three-phase switch is set to the third phase, operation of setting the location of the RCM point may terminate.

The second manipulation unit 212 may receive a command for adjusting the location of the RCM point. For this, the second manipulation unit 212 may be implemented as a stick including a 6-axis force/torque (F/T) sensor. The operator may hold the stick with his or her hand, and may apply a force to the stick to thus input a command for adjusting the location of the RCM point.

The stick may be in the shape of a cylinder or a faceted cylinder. The width or height of the stick may be designed to be appropriate values such that the operator can hold the stick with his or her hand. For example, the stick may be implemented such that when a force from the operator is applied to the stick, no mechanical movement occurs. As another example, the stick may be implemented such that when a force from the operator is applied to the stick, mechanical movement occurs in a direction in which the force is applied.

The 6-axis F/T sensor may detect forces and torques applied in the directions of three axes of x-, y-, and z-axes. The output signals of the 6-axis F/T sensor may include three force signals Fx, Fy, and Fz and three torque signals Tx, Ty, and Tz. The output signals of the 6-axis F/T sensor may be provided to the controller 220 of the slave robot 200.

FIGS. 2 and 3 show the case in which the manipulation unit 210 is disposed near the surgical instrument assembly 90, for example, on the third link 80. However, the location of the manipulation unit 210 is not limited to this. That is, the manipulation unit 210 may be disposed at an arbitrary location on the slave robot 200. As another example, the manipulation unit 210 may be disposed on another device (for example, a remote control device) separated from the slave robot 200.

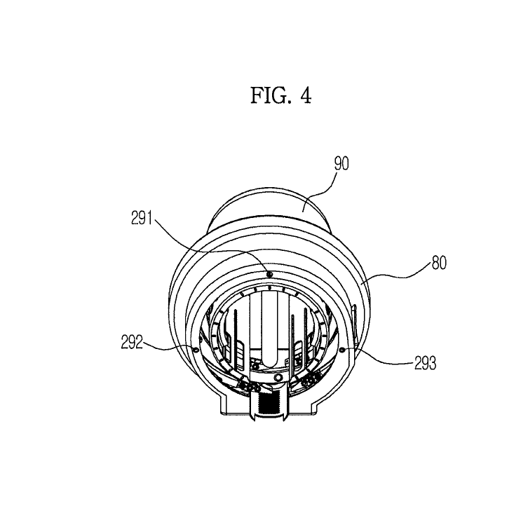

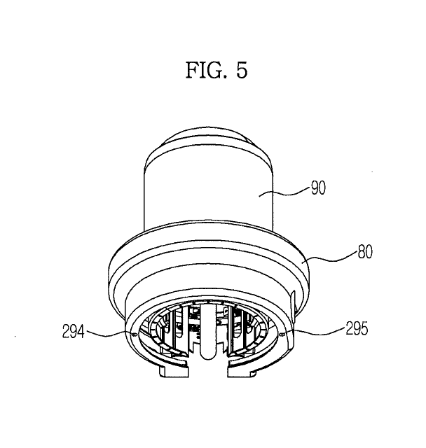

Meanwhile, a plurality of light-emitting units for indicating the RCM point may be provided on the lower end of the third link 80. The plurality of light-emitting units may be arranged at regular intervals along the circumference of the third link 80 on the lower end of the third link 80. For example, if the slave robot 200 includes three light-emitting units 291, 292, and 293 (respectively referred to as first, second, and third light-emitting units 291, 292, and 293), the three light-emitting units 291, 292, and 293 may be spaced at intervals of 120 degrees, as shown in FIG. 4. As another example, if the slave robot 200 includes two light-emitting units 294 and 295, the two light-emitting units 294 and 295 may be spaced at intervals of 180 degrees, as shown in FIG. 5. The following description, the case in which three light-emitting units are provided is assumed.

Since the plurality of light-emitting units 291, 292, and 293 may be provided for indicating the RCM point, the light-emitting units 291, 292, and 293 may be implemented as light-emitting devices for emitting light having excellent linearity, for example, laser beams. However, the light-emitting units 291, 292, and 293 are not limited to light-emitting devices for emitting laser beams, and other light-emitting devices capable of emitting arbitrary light having excellent linearity may be used as the light-emitting units 291, 292, and 293.

The light-emitting units 291, 292, and 293 may be designed such that laser beams emitted from the light-emitting units 291, 292, and 293 intersect with each other at the RCM point. Here, there will be various methods to cause an operator located near the slave robot to be able to recognize the RCM point.

For example, the light-emitting units 291, 292, and 293 may emit different colors of laser beams. If the slave robot 200 includes three light-emitting units 291, 292, and 293, the light-emitting units 291, 292, and 293 may be implemented to emit a red laser beam, a green laser beam, and a blue laser beam, respectively. In this case, the location (that is, the RCM point) at which the three colors of laser beams intersect with each other may appear as a white color.

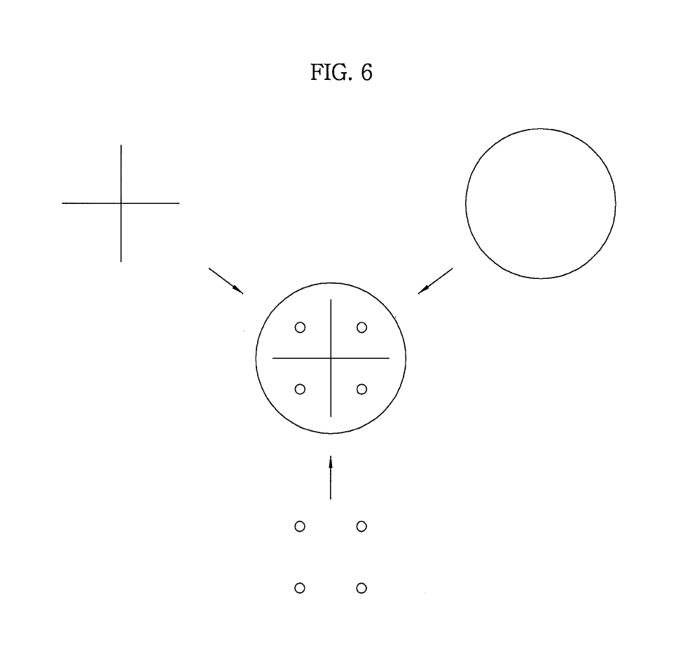

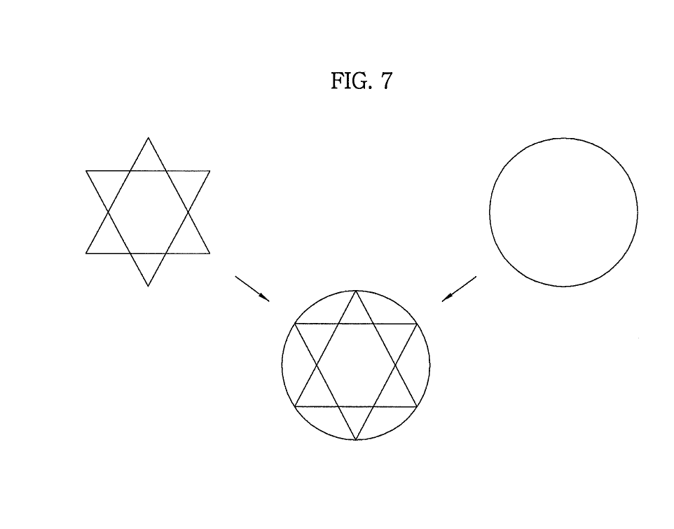

As another example, the light-emitting units 291, 292, and 293 may emit the same color of laser beams having different shapes. For this, filters having different shapes of holes may be provided in front of the respective light-emitting units 291, 292, and 293. In the filters, the remaining areas except for the holes may be opaque. Accordingly, a part of a laser beam emitted from each of the light-emitting units 291, 292, and 293 may be blocked out by a filter located in front of the corresponding light-emitting unit, and the remaining part of the laser beam may pass through the hole of the filter. The laser beam passing through the hole may have the same shape as the hole of the filter. FIG. 6 shows the case in which laser beams passed through the filters installed in front of the first, second, and third light-emitting units 291, 292, and 293 are in the shapes of a circle, a cross, and a group of points, respectively. FIG. 7 shows the case in which laser beams passed through the first light-emitting unit 291 and the second light-emitting unit 292 are in the shapes of a circle and a star, respectively.

As another example, the light-emitting units 291, 292, and 293 may emit different colors of laser beams having different shapes. For example, if the slave robot includes three light-emitting units 291, 292, and 293, the first light-emitting unit 291, the second light-emitting unit 292, and the third light emitting unit 293 may emit a red laser beam, a green laser beam, and a blue laser beam, respectively, and filters having different shapes of holes may be provided in front of the respective light-emitting units 291, 292, and 293.

In the following description, for convenience of description, the case in which the light-emitting units 291, 292, and 293 emit different colors of laser beams is assumed.

Now, a process of setting the location of a RCM point in a slave robot will be described with reference to FIGS. 8A through 8F.

First, it is assumed that an incision point has been made on a patient's abdomen, and a trocar (not shown) has been inserted in the incision point. The trocar may be in the shape of a cylinder in whose center portion a hollow hole is formed. A guide tube G may be inserted into the patient's abdomen through the hollow hole of the trocar.

In this state, if an operation of setting the location of a RCM point starts, the plurality of light-emitting units 291, 292, and 293 turn on. At this time, the plurality of light-emitting units 291, 292, and 293 may emit different colors of laser beams. The different colors of laser beams may intersect with each other to indicate a RCM point, as shown in FIG. 8A. Then, an operator may check the RCM point indicated by the light-emitting units 291, 292, and 293 with his or her naked eyes.

Thereafter, the operator may manipulate the first manipulation unit 211 to set the motion mode of the slave robot to the translation mode.

Successively, the operator may manipulate the second manipulation unit 212 to adjust the x and y coordinates of the RCM point. That is, as shown in FIG. 8B, the operator may adjust the x and y coordinates of the RCM point such that the RCM point is located just above the central point of a trocar. In detail, the operator may apply a force to the second manipulation unit 212 in the positive direction of the x-axis to adjust the x coordinate of the RCM point, and then may apply a force to the second manipulation unit 212 in the negative direction of the y-axis to adjust the y coordinate of the RCM point. However, the operator may first adjust the y coordinate of the RCM point, and then adjust the x coordinate of the RCM point.

Thereafter, the operator may adjust the second manipulation unit 212 to adjust the z coordinate of the RCM point. That is, as shown in FIG. 8C, the operator adjust the z coordinate of the RCM point such that the RCM point located above the central point of the trocar matches the central point of the trocar. In detail, the operator may apply a force to the second manipulation unit 212 in the negative direction of the z axis to adjust the z coordinate of the RCM point.

The above description with reference to FIGS. 8A, 8B, and 8C relate to the case of adjusting the z coordinate of a RCM point after adjusting the x and y coordinates of the RCM point. However, the process of adjusting the location of a RCM point is not necessarily performed in this order. Which one among the x, y, and z coordinates of a RCM point has to be first adjusted may depend on the initial location of the RCM point. In detail, FIG. 8A shows the case in which the initial location of a RCM point is above a trocar. However, unlike FIG. 8A, if the initial location of a RCM point is under a trocar, the operator may first adjust the z coordinate of the RCM point. That is, the operator may adjust the z coordinate of a RCM point such that the RCM point is located on a trocar, and then may adjust at least one of the x and y coordinates of the RCM point such that the location of the RCM point matches the central point of the trocar.

The operator may adjust the location of the RCM point while seeing it with his or her naked eyes, until the location of the RCM point matches the central point of the trocar.

If the location of the RCM point matches the central point of the trocar, the operation of setting the location of the RCM point may terminate. According to an embodiment, although the location of the RCM point has been completely set, the light-emitting units 291, 292, and 293 may continue to emit laser beams. In this case, the operator may check whether the location of the RCM point is deviated from the central point of the trocar due to rotation of the surgical instrument assembly 90. According to another embodiment, if the location of the RCM point is completely set, the light-emitting units 291, 292, and 293 may turn off.

If the location of the RCM point is completely set, as shown in FIG. 8D, the guide tube G of the surgical instrument assembly 90 may be inserted into the trocar. The guide tube G may enable a plurality of robotic surgical instruments to be inserted into a patient's abdomen. Motion of the guide tube G may be actively controlled by an actuator. A method of inserting surgical instruments into a patient's abdomen may be to put a guide tube G in a patient's abdomen, then to secure the guide tube G, to insert robotic surgical instruments in the guide tube G, and to move the robotic surgical instruments along the inner wall of the guide tube G until the robotic surgical instruments arrives at a target site. Or, there may be a method of putting a guide tube G into which robotic surgical instruments have been already inserted in a patient's abdomen.

After the guide tube G and the surgical instruments are inserted into the patient's abdomen, as shown in FIG. 8D, the surgical instrument assembly 90 may rotate in at least one direction of a roll direction, a pitch direction, and a yaw direction. FIG. 8E shows the case in which the surgical instrument assembly 90 rotates in the roll direction. FIG. 8F shows the case in which the surgical instrument assembly 90 rotates in the roll direction and the pitch direction. Since the location of the RCM point has matched the central point of the trocar, although the surgical instrument assembly 90 rotates as shown in FIGS. 8E and 8F during surgery, the patient's incision point may be prevented from being damaged due to the rotation of the surgical instrument assembly 90. Also, even after the location of the RCM point is completely set, the light-emitting units 291, 292, and 293 may be maintained in the turned-on state. Accordingly, when the location of the RCM point is deviated from the set location, the operator may recognize it and take an appropriate action.

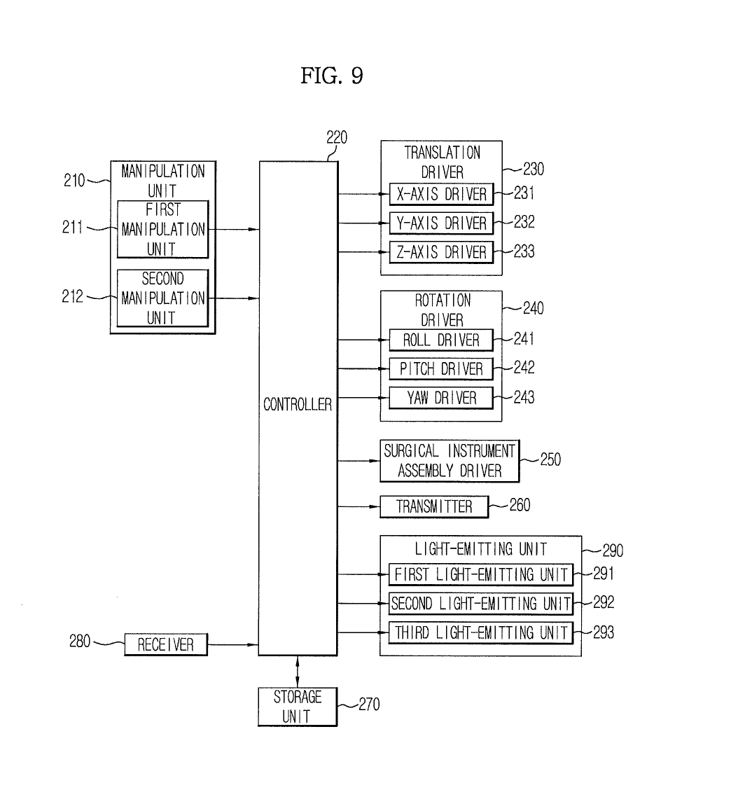

FIG. 9 is a block diagram showing the control configuration of a slave robot 200 according to one or more embodiments.

As shown in FIG. 9, the slave robot 200 may include a manipulation unit 210, a controller 220, a translation driver 230, a rotation driver 240, a surgical instrument assembly driver 250, a transmitter 260, a storage unit 270, a receiver 280, and a light-emitting unit 290.

The manipulation unit 210 may be used to allow an operator to input a command for setting the location of a RCM point of the slave robot 200. As described above, the manipulation unit 210 may include a first manipulation unit 211 for allowing an operator to input a command for switching the motion mode of the slave robot 200, and a second manipulation unit 212 for allowing an operator to input a command for adjusting the location of the RCM point.

The translation driver 230 may be in charge of the translation motion of the slave robot 200. The translation driver 230 may include a x-axis driver 231, a y-axis driver 232, and a z-axis driver 233. The x-axis driver 231, the y-axis driver 232, and the z-axis driver 233 have been described above with reference to FIG. 3, and accordingly, a repeated description will be omitted.

The rotation driver 240 may be in charge of the rotation motion of the slave robot 200. The rotation driver 240 may include a roll driver 241, a pitch driver 242, and a yaw driver 243. As described above with reference to FIGS. 2 and 3, the roll driver 241 and the pitch driver 242 may be provided in the second joint 50, and the yaw driver may be provided in the third joint 70.

The surgical instrument assembly driver 250 may provide driving force to the surgical instrument assembly 90. In detail, the surgical instrument assembly driver 250 may provide driving force to surgical instruments included in a cylindrical casing to enable the surgical instruments to be inserted into a patient's incision point through a guide tube G.

The transmitter 260 may transmit image data acquired by an endoscope or data regarding the operating state of the slave robot 200 to the master console 100.

The receiver 280 may receive a control signal from the master console 100. The received control signal is provided to the controller 220.

The controller 220 may set the location of a RCM point according to a command received through the manipulation unit 210. In detail, if setting of a RCM point location starts, the controller 220 may turn on the light-emitting unit 290. Then, for example, different colors of laser beams may be emitted from the light-emitting unit 290.

Thereafter, the controller 220 may receive an input signal from the first manipulation unit 211, and may determine the kind of the received input signal.

If it is determined that the input signal received from the first manipulation unit 211 is a signal for setting the motion mode of the slave robot 200 to a translation mode, the controller 220 may drive the translation driver 230 according to an input signal received from the second manipulation unit 212. At this time, since the motion mode of the slave robot 200 has already been set to the translation mode, no rotation motion may occur in the slave robot 200 although the operator manipulates the second manipulation unit 212.

If it is determined that the input signal received from the first manipulation unit 211 is a signal for setting the motion mode of the slave robot 200 to a rotation mode, the controller 220 may drive the rotation driver 240 according to an input signal received from the second manipulation unit 212. At this time, since the motion mode of the slave robot 200 has already been set to the rotation mode, no translation motion may occur in the slave robot 200 although the operator manipulates the second manipulation unit 211.

If setting of the RCM point location is completed, the controller 220 may turn off the light-emitting unit 290.

Thereafter, the controller 220 may control the individual components of the slave robot 200 according to a control signal received from the master console 100. For example, the controller 220 may insert the guide tube G into a patient's incision point, or insert a plurality of surgical instruments into the guide tube G.

The storage unit 270 may store information, data, an algorithm, etc. required for controlling the operation of the slave robot 200. For example, the storage unit 270 may store information or data required for setting the location of a RCM point. The storage unit 270 may be implemented, for example, as at least one of a non-volatile memory, a volatile memory, and a storage medium. However, the storage unit 270 is not limited to the above-mentioned devices, and may be implemented as another arbitrary device known to those skilled in the related field.

The light-emitting unit 290 may include a first light-emitting unit 291, a second light-emitting unit 292, and a third light-emitting unit 293. The individual light-emitting units 291, 292, and 293 may, for example, emit different colors of laser beams. Laser beams emitted from the individual light-emitting units 291, 292, and 293 may intersect with each other at a RCM point.

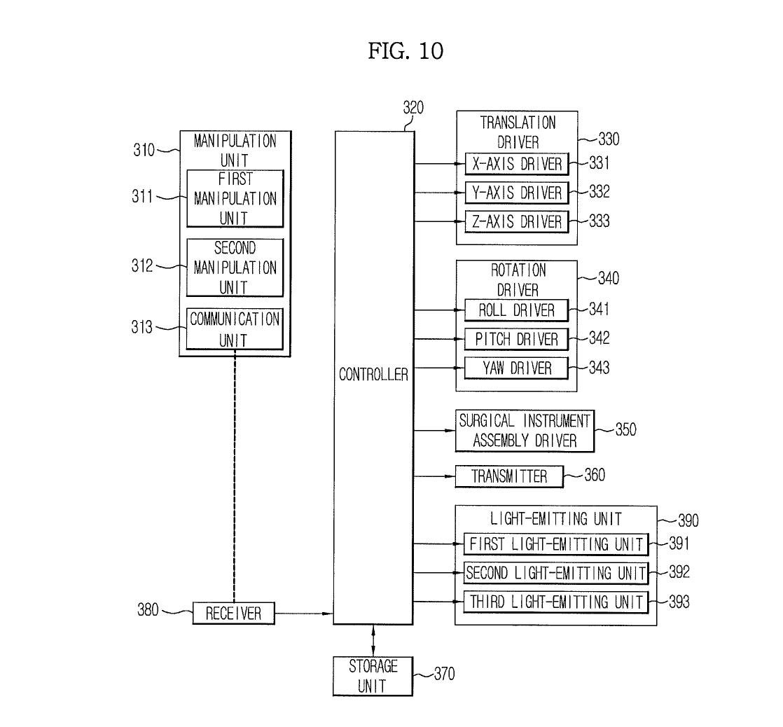

FIG. 10 is a block diagram showing the control configuration of a slave robot according to one or more embodiments.

As shown in FIG. 10, the slave robot may include a manipulation unit 310, a controller 320, a translation driver 330, a rotation driver 340, a surgical instrument assembly driver 350, a transmitter 360, a storage unit 370, a receiver 380, and a light-emitting unit 390.

The components of the slave robot shown in FIG. 10 are the same as those of the slave robot 200 shown in FIG. 9. However, FIG. 9 shows the case in which the manipulation unit 210 is integrated into the slave robot 200, whereas FIG. 10 shows the case in which the manipulation unit 310 is separated from the slave robot. As such, if the manipulation unit 310 is separated from the slave robot, the manipulation unit 310 may further include a communication unit 313, in addition to the first and second manipulation units 311 and 312.

The communication unit 313 may function to transmit input signals input through the first and second manipulation units 311 and 312 to the slave robot. At this time, the communication unit 313 may transmit input signals input through the first manipulation unit 311 or the second manipulation unit 312 to the slave robot through wireless/wired communication.

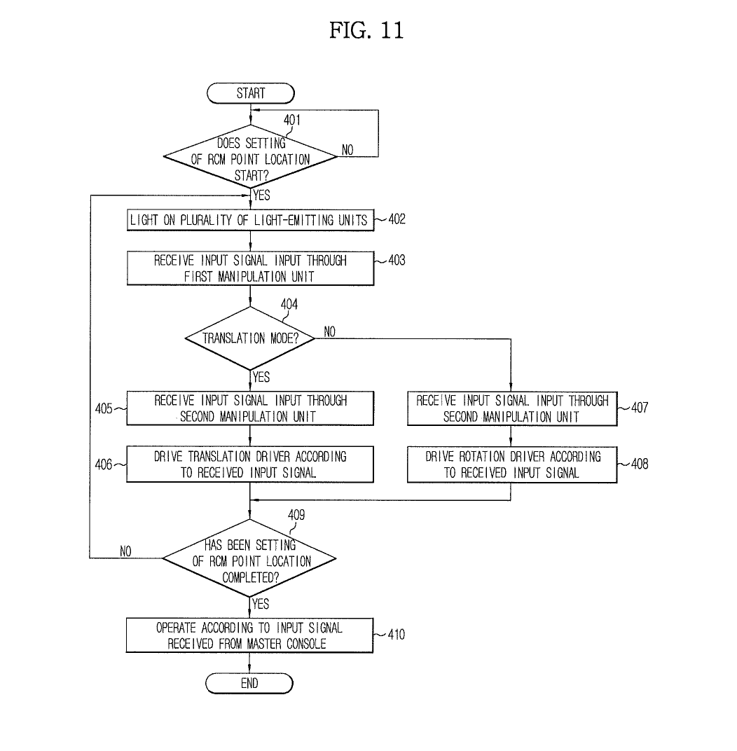

FIG. 11 is a flowchart showing a control method of the slave robot shown in FIG. 9 or 10, according to one or more embodiments.

Referring to FIGS. 9 and 11, first, the controller 220 may determine whether setting of a RCM point location starts (401).

For example, the determination may be performed based on whether a command for starting setting of a RCM point location is input. The command for starting setting of the RCM point location may be received from the master console 100 (see FIG. 1).

As another example, the determination may be performed based on whether the phase of the first manipulation unit 211 changes. In detail, it is assumed that the first manipulation unit 211 is implemented as a three-phase switch. In this case, if the phase of the first manipulation unit 211 changes from a third phase to a first phase, the controller 220 may determine that a command for starting setting of a RCM point location is input, and start setting of a RCM point location.

If setting of a RCM point location starts, the controller 220 may turn on the light-emitting units 291, 292, and 293 (402). The light-emitting units 291, 292, 293 may emit, for example, different colors of laser beams, and the emitted laser beams may intersect with each other at the RCM point.

Thereafter, an input signal input through the first manipulation unit 211 may be received (403). The input signal may be received by the controller 220 directly from the first manipulation unit 211 (see FIG. 9), or through the receiver 280 of the slave robot (see FIG. 10).

Thereafter, the controller 220 may determine whether the motion mode of the slave robot 200 has been set to a translation mode by the received input signal (404).

If the motion mode of the slave robot 200 has been set to the translation mode, the controller 220 may receive an input signal input through the second manipulation unit 212 (405).

Then, the translation driver 230 may be driven according to the received input signal (406). Operation 406 of driving the translation driver 230 may include an operation of driving at least one of the x-axis driver 231 and the y-axis driver 232, and an operation of driving the z-axis driver 233. The order in which the two operations are performed may depend on the initial location of the RCM point. As such, by controlling the second manipulation unit 212 to drive the translation driver 230, the operator may make the location of the RCM point match the central point of a trocar.

Then, it may be determined that setting of the RCM point location has been completed (409).

For example, the determination may be performed based on whether a command for terminating setting of a RCM point location is input. The command for terminating setting of the RCM point location may be received from the master console 100 (see FIG. 1).

As another example, the determination may be performed based on whether the phase of the first manipulation unit 211 of the slave robot 200 changes. In detail, it is assumed that the first manipulation unit 211 is implemented as a three-phase switch. In this case, if the phase of the first manipulation unit 211 changes from a first phase to a third phase, the controller 220 may determine that a command for terminating setting of a RCM point location is input, and terminate setting of a RCM point location.

If it is determined in operation 409 that setting of a RCM point location has been not yet completed, operations 402 through 408 are repeated.

If it is determined in operation 409 that setting of a RCM point location has been completed, the controller 220 may operate the slave robot 200 according to control signals received from the master console 100 (410). The control signals received from the master console 100 may include at least one of signals for controlling a surgical instrument selected from the surgical instrument assembly 90, and signals for rotating the surgical instrument assembly 90.

While the controller 220 controls the slave robot according to the control signals received from the master console, the light-emitting unit 290 may be maintained in the turned-on state. As such, if the light-emitting unit 290 may be maintained in the turned-on state during surgery, when the RCM point is deviated from the set location, an operator may recognize it. According to one or more embodiments, an operation of lighting off the light-emitting unit 290 may be added between operations 409 and 410.

Meanwhile, if it is determined in operation 404 that the motion mode of the slave robot 200 has not been set to the translation mode, that is, if the motion mode of the slave robot 200 has been set to the rotation mode (“No” in operation 404), the controller 220 may receive an input signal input through the second manipulation unit 212 (407). The input signal may be received by the controller 220 directly from the second manipulation unit 212 (see FIG. 9), or through the receiver 280 of the slave robot (see FIG. 10).

Then, the controller 220 may drive the rotation driver 240 according to the received input signal (408). Operation 408 of driving the rotation driver 240 may include an operation of driving at least one among the roll driver 241, the pitch driver 242, and the yaw driver 243. By driving the rotation driver 240, the controller 220 may rotate the surgical instrument assembly 90 in at least one direction of the roll direction, the pitch direction, and the yaw direction.

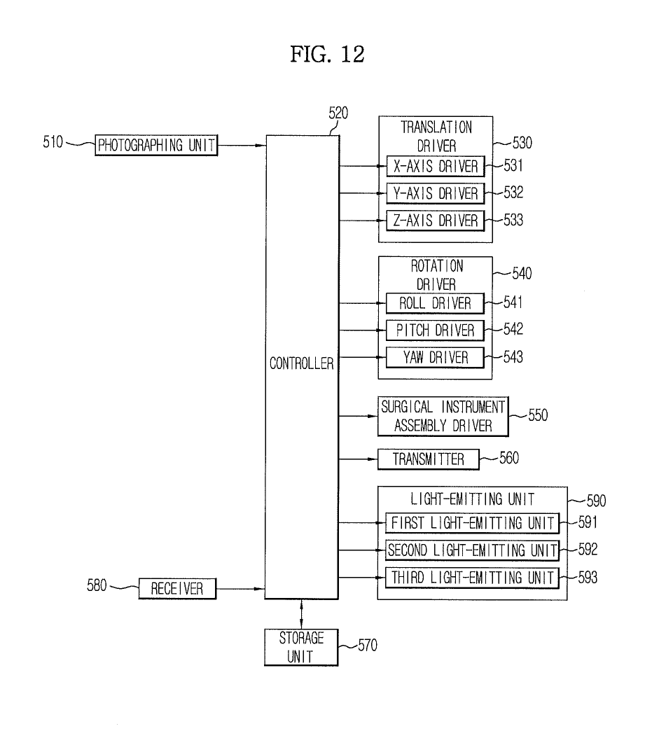

FIG. 12 is a block diagram showing the control configuration of a slave robot according to one or more embodiments.

As shown in FIG. 12, the slave robot may include a photographing unit 510, a controller 520, a translation driver 530, a rotation driver 540, a surgical instrument assembly driver 550, a transmitter 560, a storage unit 570, a receiver 580, and a light-emitting unit 590 including first, second, and third light-emitting units 591, 592, and 593.

The components of the slave robot shown in FIG. 12 are the same as those of the slave robot 200 shown in FIG. 9. However, FIG. 9 shows the case in which the manipulation unit 210 is provided in the slave robot 200, whereas FIG. 12 shows the case in which the photographing unit 510, instead of the manipulation unit 210, is provided in the slave robot.

The photographing unit 510 may be, like the light-emitting unit 290, disposed on the lower end of the third link 80 (see FIG. 2). In detail, the photographing unit 510 may be disposed to face the ground, thus acquiring images corresponding to a predetermined photographing area. The images photographed by the photographing unit 510 may include at least one among a plurality of light-emitting points, a RCM point indicated by the first, second, and third light-emitting units 591, 592, and 593, a patient's incision point, and a trocar inserted into the patient's incision point. The light-emitting points may be points at which laser beams emitted from the light-emitting units 591, 592, and 593 meet the patient's skin.

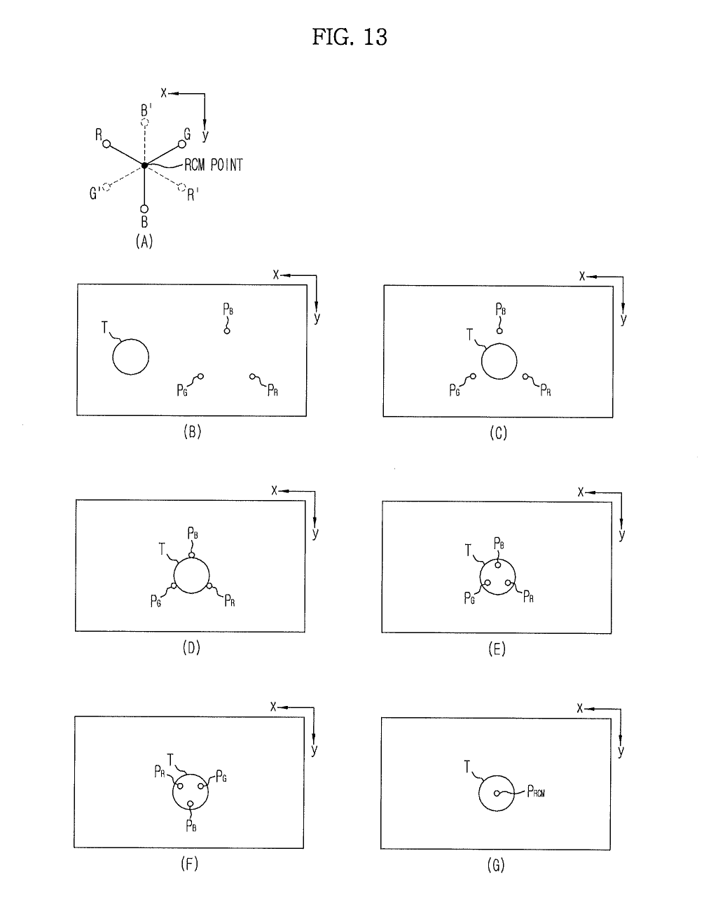

The controller 520 may analyze the images received from the photographing unit 510, and may perform setting of a RCM point location according to the results of the analysis. For this, the controller 520 may detect a trocar and a plurality of light-emitting points from the images received from the photographing unit 510. Then, the controller 520 may compare the location of the trocar to the locations of the plurality of light-emitting points. Thereafter, the controller 520 may drive the translation driver 530 according to the results of the comparison. The controller 520 may continue to analyze images photographed by the photographing unit 510, and drive the translation driver 530 according to the results of the analysis, until the location of the RCM point matches the central point of the trocar. Setting the location of a RCM point according to the results of image analysis will be described in more detail with reference to FIG. 13, below.

FIG. 13 is a view for explaining a process of setting the location of a RCM point in a slave robot according to one or more embodiments, such as the slave robot shown in FIG. 12.

(A) of FIG. 13 is a plan showing different colors of laser beams that may be emitted from the light-emitting units 591, 592, and 593, and a RCM point that may be indicated by the different colors of laser beams. (B) through (G) of FIG. 13 show images photographed by the photographing unit 510 and arranged in the order of time.

First, it is assumed that a trocar has been inserted into a patient's incision point. Also, it is assumed that the initial location of a RCM point has been deviated by a predetermined distance from the central point of the trocar, and positioned above the trocar (see FIG. 8A).

An image photographed by the photographing unit 510 just after setting of a RCM point location starts is shown in (B) of FIG. 13. The image shown in (B) of FIG. 13 includes a trocar T and a plurality of light-emitting points P_{R}, P_{G}, and P_{B}.

The controller 520 may analyze the image to detect the trocar T and the plurality of light-emitting points P_{R}, P_{G}, and P_{B}. Then, the controller 520 may compare the location of the trocar T to the locations of the light-emitting points P_{R}, P_{G}, and P_{B}. As the result of the comparison, the controller 520 may determine that the light-emitting points P_{R}, P_{G}, and P_{B} have been deviated by a predetermined distance from the central point of the trocar T in the negative direction of the x-axis. Accordingly, the controller 520 may drive the x-axis driver 531 of the translation driver 530 to move the x-coordinate of the RCM point in the positive direction of the x-axis. An image photographed by the photographing unit 510 after moving the x-coordinate of the RCM point is shown in (C) of FIG. 13.

Thereafter, the controller 520 may analyze the image shown in (C) of FIG. 13 to detect the trocar T and the light-emitting points P_{R}, P_{G}, and P_{B}. Then, the controller 520 may compare the location of the trocar T to the locations of the light-emitting points P_{R}, P_{G}, and P_{B}. As the result of the comparison, the controller 520 may determine that the light-emitting points P_{R}, P_{G}, and P_{B} are located outside the trocar T. Also, the controller 520 may determine that the locations of the light-emitting points P_{R}, P_{G}, and P_{B} are opposite to the locations of the corresponding light-emitting units 591, 592, and 593. Thus, the controller 520 may determine that the location of the RCM point is above the trocar T. Accordingly, the controller 520 may drive the z-axis driver 533 of the translation driver 530 to move the z coordinate of the RCM point in the negative direction of the z-axis. An image photographed by the photographing unit 510 after moving the z coordinate of the RCM point is shown in (D) of FIG. 13.

Thereafter, the controller 520 may analyze the image shown in (D) of FIG. 13 to detect the trocar T and the light-emitting points P_{R}, P_{G}, and P_{B}. Then, the controller 520 may compare the location of the trocar T to the locations of the light-emitting points P_{R}, P_{G}, and P_{B}. As the result of the comparison, the controller 520 may determine that the light-emitting points P_{R}, P_{G}, and P_{B} are located on the boundary of the trocar T. Also, the controller 520 may determine that the locations of the light-emitting points P_{R}, P_{G}, and P_{B} are opposite to the locations of the corresponding light-emitting units 591, 592, and 593. Thus, the controller 520 may determine that the location of the RCM point is still above the trocar T. Accordingly, the controller 520 may drive the z-axis driver 533 of the translation driver 530 to move the z coordinate of the RCM point in the negative direction of the z axis. An image photographed by the photographing unit 510 after moving the z coordinate of the RCM point is shown in (E) of FIG. 13.

Then, the controller 520 may analyze the image shown in (E) of FIG. 13 to detect the trocar T and the light-emitting points P_{R}, P_{G}, and P_{B}. Then, the controller 520 may compare the location of the trocar T to the light-emitting points P_{R}, P_{G}, and P_{B}. As the result of the comparison, the controller 520 may determine that the light-emitting points P_{R}, P_{G}, and P_{B} are located inside the boundary of the trocar T. Also, the controller 520 may determine that the locations of the light-emitting points P_{R}, P_{G}, and P_{B} are opposite to the locations of the corresponding light-emitting units 591, 592, and 593. Thus, the controller 520 may determine that the location of the RCM point is still above the trocar T. Accordingly, the controller 520 may drive the z-axis driver 533 of the translation driver 530 to move the z coordinate of the RCM point in the negative direction of the z axis. An image photographed by the photographing unit 510 after moving the z coordinate of the RCM point is shown in (F) of FIG. 13.

Thereafter, the controller 520 may analyze the image shown in (F) of FIG. 13 to detect the trocar T and the light-emitting points P_{R}, P_{G}, and P_{B}. Then, the controller 520 may compare the location of the trocar T to the light-emitting points P_{R}, P_{G}, and P_{B}. As the result of the comparison, the controller 520 may determine that the light-emitting points P_{R}, P_{G}, and P_{B} are located inside the boundary of the trocar T. Also, the controller 520 may determine that the locations of the light-emitting points P_{R}, P_{G}, and P_{B} are the same as those of the corresponding light-emitting units 591, 592, and 593. Thus, the controller 520 may determine that the location of the RCM point is under the trocar T. Accordingly, the controller 520 may drive the z axis driver 533 of the translation driver 530 to move the z coordinate of the RCM point in the positive direction of the z-axis. An image photographed by the photographing unit after moving the z coordinate of the RCM point is shown in (G) of FIG. 13.

Thereafter, the controller 520 may analyze the image shown in (G) of FIG. 13 to detect the trocar T and the light-emitting points P_{R}, P_{G}, and P_{B}. Then, the controller 520 may compare the location of the trocar T to the light-emitting points P_{R}, P_{G}, and P_{B}. As the result of the comparison, the controller 520 may determine that only one light-emitting point has been detected. Since only one light-emitting point has been detected, the controller 520 may determine the detected light-emitting point to be a RCM point P_{RCM}. Also, the controller 520 may determine that the RCM point P_{RCM} matches the central point of the trocar T. Thus, the controller 520 may determine that the location of the RCM point matches the central line of the trocar T. Accordingly, the controller 520 may terminate setting of a RCM point location.

Even after setting of the RCM point location terminates, the light-emitting units 591, 592, and 593 may be maintained in the turned-on state. Thereby, when the location of the RCM point is deviated from the set location, the operator may recognize it and take an appropriate action.

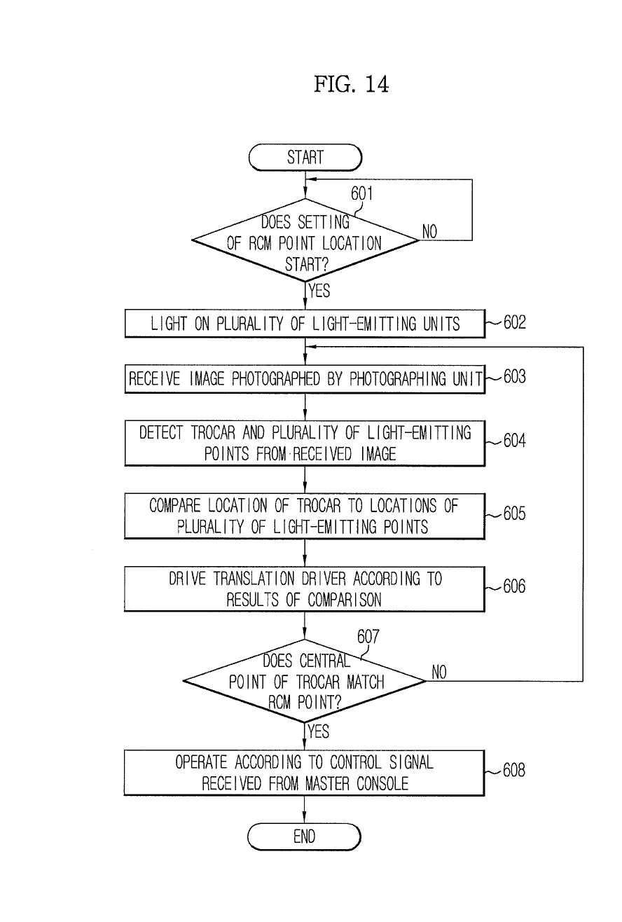

FIG. 14 is a flowchart showing a control method of the slave robot shown in FIG. 12, according to one or more embodiments.

First, the controller 520 may determine whether setting of a RCM point location starts (601). The determination may be performed based on whether a command for starting setting of a RCM point location is input. The command for starting setting of a RCM point location may be received from the master console 100 (see FIG. 1).

If setting of a RCM point location starts (“Yes” in operation 601), the controller 520 may turn on the light-emitting units 591, 592, and 593 (602). Then, the light-emitting units 591, 592, and 593 may, for example, different colors of laser beams, and the laser beams may intersect with each other at a RCM point.

Thereafter, the controller 520 may receive an image photographed by the photographing unit 510 (603).

Then, the controller 520 may detect a trocar and a plurality of light-emitting points from the received image (604).

Then, the controller 520 may compare the location of the trocar to the locations of the light-emitting points (605), and may drove the translation driver 530 according to the results of the comparison (606).

Successively, the controller 520 may receive an image photographed by the photographing unit 510, and may analyze the received image to determine whether the central point of the trocar matches the RCM point (607).

If it is determined in operation 607 that the central point of the trocar does not match the RCM point (“No” in operation 607), operations 603 through 606 may be repeated. For example, if a plurality of light-emitting points have been detected from the photographed image, the controller 520 may determine that the RCM point does not match the central point of the trocar. Also, if although only one light-emitting point, that is, a RCM point has been detected from the photographed image, the location of the detected RCM point does not match the central point of the trocar, the controller 520 may repeat operations 603 through 606.

If it is determined in operation 607 that the central point of the trocar matches the RCM point (“Yes” in operation 607), the controller 520 may terminate setting of a RCM point location. Then, the controller 520 may operate the slave robot according to a control signal received from the master console 100 (608).

In one or more embodiments, any apparatus, system, element, or interpretable unit descriptions herein include one or more hardware devices or hardware processing elements. For example, in one or more embodiments, any described apparatus, system, element, retriever, pre or post-processing elements, tracker, detector, encoder, decoder, etc., may further include one or more memories and/or processing elements, and any hardware input/output transmission devices, or represent operating portions/aspects of one or more respective processing elements or devices. Further, the term apparatus should be considered synonymous with elements of a physical system, not limited to a single device or enclosure or all described elements embodied in single respective enclosures in all embodiments, but rather, depending on embodiment, is open to being embodied together or separately in differing enclosures and/or locations through differing hardware elements.

In addition to the above described embodiments, embodiments can also be implemented through computer readable code/instructions in/on a non-transitory medium, e.g., a computer readable medium, to control at least one processing device, such as a processor or computer, to implement any above described embodiment. The medium can correspond to any defined, measurable, and tangible structure permitting the storing and/or transmission of the computer readable code.

The media may also include, e.g., in combination with the computer readable code, data files, data structures, and the like. One or more embodiments of computer-readable media include: magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Computer readable code may include both machine code, such as produced by a compiler, and files containing higher level code that may be executed by the computer using an interpreter, for example. The media may also be any defined, measurable, and tangible distributed network, so that the computer readable code is stored and executed in a distributed fashion. Still further, as only an example, the processing element could include a processor or a computer processor, and processing elements may be distributed and/or included in a single device.

The computer-readable media may also be embodied in at least one application specific integrated circuit (ASIC) or Field Programmable Gate Array (FPGA), as only examples, which execute (e.g., processes like a processor) program instructions.

While aspects of the present invention has been particularly shown and described with reference to differing embodiments thereof, it should be understood that these embodiments should be considered in a descriptive sense only and not for purposes of limitation. Descriptions of features or aspects within each embodiment should typically be considered as available for other similar features or aspects in the remaining embodiments. Suitable results may equally be achieved if the described techniques are performed in a different order and/or if components in a described system, architecture, device, or circuit are combined in a different manner and/or replaced or supplemented by other components or their equivalents.

Thus, although a few embodiments have been shown and described, with additional embodiments being equally available, it would be appreciated by those skilled in the art that changes may be made in these embodiments without departing from the principles and spirit of the invention, the scope of which is defined in the claims and their equivalents.

What is claimed is:

1. A single port surgical robot comprising:
a first link connected to a body by a first joint in a direction perpendicular to the body, the first link having a linear structure;
a second link connected to an upper end of the first link by a second joint, the second link having a curved structure;
a third link connected to an upper end of the second link by a third joint, the third link having a cylindrical structure;
a plurality of light-emitting units arranged on a lower end of the third link along the circumference of the third link, and configured to emit light toward a remote center motion (RCM) point; and
a controller configured to adjust the location of the RCM point.

2. The single port surgical robot according to claim 1, further comprising a manipulation unit,
wherein the manipulation unit comprises a first manipulation unit configured to set a motion mode of a surgical instrument assembly connected to an upper part of the third link to a translation mode or a rotation mode, and a second manipulation unit configured to receive a command for translating the surgical instrument assembly or a command for rotating the surgical instrument assembly based on the RCM point.

3. The single port surgical robot according to claim 2, wherein the second manipulation unit includes a force/torque (F/T) sensor for sensing a force applied by an operator, and the controller adjusts the location of the RCM point according to a command input through the second manipulation unit.

4. The single port surgical robot according to claim 2, wherein the manipulation unit is separated from the single port surgical robot.

5. The single port surgical robot according to claim 1, wherein the plurality of light-emitting units emit different colors of light.

6. The single port surgical robot according to claim 1, wherein a plurality of filters having different shapes of holes are provided in front of the respective light-emitting units.

7. The single port surgical robot according to claim 1, further comprising a photographing unit disposed on the lower end of the third link, and configured to photograph an image corresponding to a photographing area,
wherein the photographed image includes the RCM point, a trocar inserted into a patient's incision point, and at least one among a plurality of light-emitting points created by the plurality of light-emitting units.

8. The single port surgical robot according to claim 7, wherein the controller detects at least one among the trocar, the plurality of light-emitting points, and the RCM point from the photographed image, and automatically adjusts the location of the RCM point based on the result of the detection.

9. The single port surgical robot according to claim 8, wherein if the detected light-emitting points are located outside the trocar, the controller adjusts at least one of the x and y coordinates of the RCM point.

10. The single port surgical robot according to claim 8, wherein the controller adjusts the z coordinate of the RCM point, according to the results of comparison between the detected light-emitting points and the locations of the light-emitting units.

11. A control method of a single port surgical robot, comprising:
operating a plurality of light-emitting units arranged along the circumference of a cylindrical link, and configured to emit light toward a remote center motion (RCM) point; and
adjusting the location of the RCM point.

12. The control method according to claim 11, further comprising receiving a command for setting a motion mode of a surgical instrument assembly connected to an upper part of the link to a translation mode, from a first manipulation unit.

13. The control method according to claim 12, wherein the adjusting of the location of the RCM point comprises translating the surgical instrument assembly according to a command input through a second manipulation unit, to thus adjust the location of the RCM point, the second manipulation unit including a force/torque (F/T) sensor for sensing a force applied by an operator.

14. The control method according to claim 11, further comprising photographing an image corresponding to a photographing area using a photographing unit disposed on the lower end of the link,
wherein the photographed image includes at least one among the RCM point, a trocar inserted into a patient's incision point, and a plurality of light-emitting points created by the plurality of light-emitting units.

15. The control method according to claim 14, wherein the adjusting of the location of the RCM point comprises:
detecting at least one among the trocar, the plurality of light-emitting points, and the RCM point from the photographed image; and
automatically adjusting the location of the RCM point based on the results of the detection.

16. A single port surgical robot comprising:
a plurality of light-emitting units emitting highly linear beams and configured such that the beams emitted from the plurality of light-emitting units intersect with each other at the RCM point; and
a controller configured to adjust the location of the RCM point.

17. The single port surgical robot according to claim 16, further comprising:
a first link connected to a body by a first joint in a direction perpendicular to the body, the first link having a linear structure;
a second link connected to an upper end of the first link by a second joint, the second link having a curved structure; and
a third link connected to an upper end of the second link by a third joint, the third link having a cylindrical structure,
wherein the plurality of light-emitting units are arranged on a lower end of the third link along the circumference of the third link.

18. The single port surgical robot according to claim 16, further comprising a manipulation unit,
wherein the manipulation unit is configured to adjust the location of the RCM point based on a command input by an operator.

19. The single port surgical robot according to claim 16, further comprising a photographing unit configured to photograph an image corresponding to a photographing area,
wherein the photographed image includes the RCM point, a trocar inserted into a patient's incision point, and at least one among a plurality of light points created by the plurality of light-emitting units.

20. The single port surgical robot according to claim 19, wherein the controller detects at least one among the trocar, the plurality of light-emitting points, and the RCM point from the photographed image, and automatically adjusts the location of the RCM point based on the result of the detection.
